(19) United States
(12) Patent Application Publication
(10) Pub. No.: US 2002/0052332 A1
(43) Pub. Date: May 2, 2002

(54) Methods of inhibiting retinal pigment epithelial cell degeneration, stable cell lines, and uses

(75) Inventor: Bernard F. Godley, Kemah, TX (US)

Correspondence Address: MUETING, RAASCH & GEBHARDT, P.A., P.O. BOX 581415, MINNEAPOLIS, MN, 55458, US

(73) Assignee: The Board of Regents, University of Texas System

(21) Appl. No.: 09/832,611

(22) Filed: Apr. 11, 2001

Related U.S. Application Data
(60) Provisional application No. 60/196,218, filed on Apr. 11, 2000.

(51) Int. Cl. A61K 31/70; A01N 43/04; C12P 21/06; C12N 15/00; C12N 15/09; C12N 15/63; C12N 15/70; C12N 15/74

(52) U.S. Cl. 514/44; 435/69.1; 435/320.1

(57) ABSTRACT

The present invention provides methods for inhibiting the degeneration of retinal pigment epithelial cells, which can reduce the incidence of, or delay the onset of, age-related macular degeneration, particularly in the early stages of AMD or in individuals at high risk for the disease.

### CROSS-REFERENCE TO RELATED APPLICATION

The present invention claims priority to Provisional Application Ser. No. 60/196,218, filed on Apr. 11, 2000, which is incorporated herein by reference.

### GRANT INFORMATION

The present invention was made with the support of Grant No. EY12850-01 from the National Institutes of Health. The government may have certain rights in this invention.

### BACKGROUND OF THE INVENTION

The retinal pigment epithelial (RPE) layer plays a key role in sustaining the health and functional integrity of the neurosensory retina. RPE dysfunction and death is linked to acquired diseases of the retina, such as age-related macular degeneration (AMD). AMD is a leading cause of catastrophic central visual impairment (irreversible blindness) for which the cause is uncertain and for which no preventive or curative treatment exists. In fact, AMD is one of the leading causes of blindness in older adults in the United States, and may account for up to 30% of all bilateral blindness among Caucasian Americans.

It is believed that AMD results from a combination of intrinsic factors and extrinsic factors that result in RPE dysfunction and cell death yielding the clinical manifestations of AMD. While there is indirect evidence supporting a genetic influence on susceptibility, it is also apparent that oxidative stress may play an important role; however, the cellular and molecular consequences of oxidative stress on RPE are not well defined. One working hypothesis is that during the aging process, reactive oxygen intermediates (ROI) overwhelm declining RPE antioxidant defenses leading to mitochondrial DNA (mtDNA) damage, cell dysfunction, and death. The conceptual basis for this hypothesis results from evidence that: 1) RPE cells are continuously exposed to oxidative stress; 2) antioxidant defenses decline with age and may be lower in AMD patients compared to controls; and 3) mtDNA damage occurs preferentially in ROI-treated RPE cells and is repaired less efficiently and is associated with mitochondrial functional decline and cell death. Thus, there is a need for methods for inhibiting degeneration of RPE cells. Also, there is a need for a more effective and permanent treatment for AMD.

### SUMMARY OF THE INVENTION

The present invention provides methods for inhibiting the degeneration of retinal pigment epithelial cells, which can reduce the incidence of, or delay the onset of, age-related macular degeneration, particularly in the early stages of AMD or in individuals at high risk for the disease. This can be accomplished by genetically engineering autologous or allograft retinal pigment epithelial cells in vitro, or significantly and preferably, by genetically engineering in situ (i.e., in vivo), retinal pigment epithelial cells to produce increased glutathione-S-transferase activity. If genetically engineered in vitro, the cells can be implanted subsequently under the retina.

In one embodiment of the invention, methods for generating genetically engineered RPE cells are provided. One method includes contacting an RPE cell with a polynucleotide that encodes a glutathione-S-transferase under conditions effective for the uptake of the polynucleotide into the RPE cell. This method can be carried out in vitro or, preferably, in vivo. Herein, “conditions effective for the uptake of the polynucleotide” include conditions that allow for the polynucleotide to transfect or transform the cell.

In another embodiment of the invention, methods for inhibiting the degeneration (e.g., death) of RPE cells are provided. One method includes incorporating an exogenous polynucleotide into an RPE cell, wherein the exogenous polynucleotide encodes a polypeptide useful in the inhibition of the degeneration of RPE cells. Preferably, this method is carried out in situ (i.e., in vivo). More preferably, the polypeptide is a glutathione-S-transferase enzyme.

In another embodiment of the invention, methods for treating age-related macular degeneration in a subject are provided. One method includes incorporating an exogenous polynucleotide into an in vivo RPE cell, wherein the exogenous polynucleotide encodes a polypeptide useful in the treatment of age-related macular degeneration. Preferably, the polypeptide is a glutathione-S-transferase enzyme.

In yet another embodiment, there is provided a method of delivering a polynucleotide to a subject's eye. The method involves delivering the polynucleotide to the subretinal space of the subject's eye. Preferably, the polynucleotide is delivered by injection of a liquid that includes the polynucleotide.

In this context, the terms “treatment” or “treating” refer to preventing, delaying the onset of, or reversing age-related macular degeneration, particularly in the early stages of AMD or in individuals at high risk for the disease. Thus, this includes both therapeutic and prophylactic methods of treatment.

A “subject” includes both humans and other animals and organisms. Thus, the methods are applicable to both human therapy and veterinary applications. For example, the veterinary applications include, but are not limited to, canine, bovine, feline, porcine, equine, and ovine animals, as well as other domesticated animals including reptiles, birds, rabbits, and rodents such as rats, mice, guinea pigs and hamsters. Valuable non-domesticated animals, such as zoo animals, may also be treated. In the preferred embodiment, the animal is a mammal, and in the most preferred embodiment the animal is human.

The present invention also provides a method of inhibiting damage to a retinal pigment epithelial cell upon exposure to reactive oxygen intermediates. The present invention also provides a method of inhibiting damage to mitochondrial DNA in a retinal pigment epithelial cell upon exposure to reactive oxygen intermediates. These methods include incorporating a Bcl-2 gene into a retinal pigment epithelial cell. In this context, “inhibiting” means reducing the amount of damage, preventing damage, and/or reversing damage.

The present invention also provides RPE cells containing an exogenous polynucleotide that encodes a glutathione-S-transferase.

As used in the specification and in the claims, “a” or “an” can mean one or more, depending upon the context in which it is used. Thus, for example, reference to “a cell” means that at least one cell is utilized.

The term “in situ” refers to a retinal pigment epithelial cell contained within the eye, i.e., in vivo.

The term “genetically engineered” refers to a cell or tissue that has been subjected to recombinant nucleic acid manipulations, such as the introduction of an exogenous polynucleotide, resulting in a cell or tissue that is in a form not ordinarily found in nature. Generally, the exogenous polynucleotide is made using recombinant nucleic acid techniques. It is understood by one skilled in the art that once a genetically engineered cell or tissue is made, it may replicate nonrecombinantly, i.e., using the in vivo cellular machinery of the host cell, but will still be considered genetically engineered for the purposes of the invention. Thus, genetically engineered cells or tissues can result from manipulations that cause upregulation of endogenous glutathione-S-transferase or increased glutathione-S-transferase activity, for example.

“Polynucleotide” as used herein refers to a polymeric form of nucleotides of any length, either ribonucleotides or deoxynucleotides, and includes both double-and single-stranded DNA and RNA. A polynucleotide may include both coding and noncoding regions, and can be obtained directly from a natural source (e.g., a microbe), or can be prepared with the aid of recombinant, enzymatic, or chemical techniques. A polynucleotide can be linear or circular in topology. A polynucleotide can be, for example, a portion of a vector, such as an expression or cloning vector, or a fragment.

“Polypeptide” as used herein refers to a polymer of amino acids and does not refer to a specific length of a polymer of amino acids. Thus, for example, the terms peptide, oligopeptide, protein, and enzyme are included within the definition of polypeptide. This term also includes post-expression modifications of the polypeptide, for example, glycosylations, acetylations, phosphorylations and the like.

“Coding region” refers to a polynucleotide that encodes a polypeptide, usually via mRNA, when placed under the control of appropriate regulatory sequences. The boundaries of the coding region are generally determined by a translation start codon at its 5′ end and a translation stop codon at its 3′ end. “Exogenous coding region” refers to a foreign coding region, i.e., a coding region that is not normally present in a microbe, or a coding region that is normally present in a microbe but is operably linked to a regulatory region to which it is not normally operably linked.

“Regulatory region” refers to a polynucleotide that regulates expression of a coding region to which a regulatory region is operably linked. Nonlimiting examples of regulatory regions include promoters, enhancers, transcription initiation sites, translation start sites, translation stop sites, and terminators.

“Operably linked” refers to a juxtaposition wherein the components so described are in a relationship permitting them to function in their intended manner. A regulatory element is “operably linked” to a coding region when it is joined in such a way that expression of the coding region is achieved under conditions compatible with the regulatory region.

The terms “complement” and “complementary” as used herein, refer to the ability of two single stranded polynucleotides to base pair with each other, where an adenine on one polynucleotide will base pair to a thymine on a second polynucleotide, and a cytosine on one polynucleotide will base pair to a guanine on a second polynucleotide. Two polynucleotides are complementary to each other when a nucleotide sequence in one polynucleotide can base pair with a nucleotide sequence in a second polynucleotide. For instance, 5′-ATGC and 5′-GCAT are complementary. The terms complement and complementary also encompass two polynucleotides where one polynucleotide contains at least one nucleotide that will not base pair to at least one nucleotide present on a second polynucleotide. For instance the third nucleotide of each of the two polynucleotides 5′-ATTGC and 5′-GCTAT will not base pair, but these two polynucleotides are complementary as defined herein. Typically two polynucleotides are complementary if they hybridize under certain conditions.

As used herein, “hybridizes,” “hybridizing,” and “hybridization” means that a single stranded polynucleotide forms a noncovalent interaction with a complementary polynucleotide under certain conditions, as described herein.

### BRIEF DESCRIPTION OF THE DRAWINGS

FIG. 1. Effect of hydrogen peroxide on mitochondrial function in mGSTA1-1 transfected human RPE cells. Plated RPE cells were exposed to hydrogen peroxide at the indicated concentrations for 1 hour prior to MTT assay (n=5 for each group, values are mean +/−SEM, *=p<0.05 compared to nontransfected RPE cells).

FIG. 2. Effect of cumene hydroperoxide on mitochondrial function in mGSTA1-1 transfected human RPE cells. Plated RPE cells were exposed to cumene hydroperoxide at the indicated concentrations for 1 hour prior to MTT assay (n=5 for each group, values are mean +/−SEM, * p<0.05 compared to nontransfected RPE cells).

FIG. 3. Protection of mitochondrial redox function from H₂O₂ damage by Bcl-2 overexpression. Control, vector-only, and Bcl-2 transfected cultured RPE cells were exposed to different concentrations of H₂O₂ for 1 h. Mitochondrial redox function in cell lines was evaluated from the capacity to reduce MTT. The data represents four experiments. Each experiment was performed in duplicate.

FIG. 4. Protection of mitochondrial DNA against H₂O₂ induced damage by Bcl-2 overexpression. Quantitative polymerase chain reaction (QPCR) assays were employed to determine average lesion frequencies in the 13.4 kb β-globin nuclear DNA (A) and the 16.2 kb mitochondrial DNA (B) products in RPE cell lines. Black bars represent data from control cells; white bars represent data from vector-only transfected cell; and gray bars represent data obtained from Bcl-2 transfected cells. The data represents three experiments. Each experiment was performed in duplicate.

### DETAILED DESCRIPTION OF PREFERRED EMBODIMENTS OF THE INVENTION

The present invention provides methods for inhibiting the degeneration of retinal pigment epithelial cells and treating age-related macular degeneration. It is believed that this can be accomplished by protecting against low levels of chronic oxidative stress. In one aspect, the present invention accomplishes this by overexpressing a polypeptide, preferably a glutathione-S-transferase enzyme, in retinal pigment epithelial cells. In another aspect, the use of an anti-death gene of caenorhabtidis elegans, such as a Bcl-2 gene, can accomplish this same result. In a preferred embodiment, the present invention is directed to the use of genetic manipulation (e.g., gene therapy) to enhance RPE cell function and survival resulting in preservation of vision.

The glutathione-S-transferases (GST) are an important group of detoxification enzymes found in all eucaryotic species. These enzymes play important roles in providing protection against electrophiles and end products of oxidative stress. The detoxification properties of GSTs result from their ability to conjugate glutathione with compounds containing an electrophilic center. The majority of GST substrates are either xenobiotics or products of oxidative stress. It is believed that low levels of chronic oxidative stress may be physiologically more relevant compared to short interval, high-dose exposure, especially with respect to AMD.

The present invention provides stable GST-transfected RPE cell lines, which carry mouse or human gene isoforms of GST, which express the protein at higher levels than the RPE cells prior to transfection. Significantly, these cells demonstrate enhanced protection from mitochondrial dysfunction and cell death, compared to controls after exposure to oxidative stress.

The present invention also provides methods of gene transfer to human RPE cells for enhancing the antioxidant protection of human RPE cells. Preferably, this method of gene transfer can be accomplished in situ during a surgical procedure, although gene transfer can be accomplished in vitro and the cells implanted under the retina. The goal of this procedure is to protect the outer retinal layers from oxidative damage and prevent the initiation and progression of AMD. The method involves the delivery of genetic material to the RPE cell layer using equipment and techniques in common use today.

In a preferred embodiment, a method involves accessing the posterior segment of the eye, preferably through a standard pars plana vitrectomy operation, which can be performed in an inpatient or outpatient setting. The genetic material (e.g., gene, coding region of a gene, or vector), preferably in liquid form, can then be delivered to the subretinal space, using, for example a cannula (e.g., a 39 or 42 gauge cannula). A subretinal blister of this liquid is formed in the macular portion of the retina and the operation is concluded, allowing the fluid to reabsorb and the gene transfection to the RPE cell layer to occur.

Preferably, the polynucleotide used in genetically engineering the RPE cells is “exogenous” or “foreign” to the RPE cells. Thus, exogenous polynucleotides include nucleic acid that is not ordinarily found in the genome of the RPE cells, such as heterologous nucleic acid from other organisms. It also includes nucleic acid that is found in the RPE cells but not expressed in sufficient amounts for therapeutic or prophylactic treatment. Thus, as used herein, a polynucleotide is exogenous to an RPE cell if it contains a coding region for the polypeptide of interest but it is operably linked to a promoter, for example, to which it is not operably linked in nature. It is understood by one skilled in the art that once an exogenous polynucleotide is made and introduced into a host cell or organism, it will replicate non-recombinantly, i.e., using the in vivo cellular machinery of the host cell rather than in vitro manipulations; however, such polynucleotides, once produced recombinantly, although subsequently replicated nonrecombinantly, are still considered “exogenous” or “recombinant” for the purposes of the invention. Furthermore, genetically engineered cells or tissues can result from various manipulations of the genetic machinery that cause upregulation of endogenous glutathione-S-transferase or increased glutathione-S-transferase activity, for example.

Similarly, an “exogenous” or “recombinant” polypeptide is one made using recombinant techniques, i.e., through the expression of an exogeneous or recombinant polynucleotide as described above. A recombinant polypeptide is distinguished from naturally occurring polypeptide by at least one or more characteristics. For example, the polypeptide may be made at a significantly higher concentration than is ordinarily seen, through the use of a inducible promoter or high expression promoter, such that increased levels of the polypeptide are made. Thus, for instance, an exogenous polypeptide is one which is not ordinarily expressed in the RPE cells. Alternatively, the exogenous polypeptide may be in a form not ordinarily found in nature, as in the addition of an epitope tag or amino acid substitutions, insertions, and deletions. Furthermore, the exogenous polypeptide may be expressed at a level that is not ordinarily expressed in RPE cells.

Examples of suitable polynucleotides that can be incorporated into RPE cells as described herein include glutathione peroxidases, a class of enzymes that detoxify lipid hydroperoxides (e.g., GPX1 having Genbank Accession No. M21304, GPX2 having Genbank Accession No. X91863, GPX3 having Genbank Accession No. NM002084, GPX4 having Genbank Accession No. NM002085, etc.), glutathione transferases, a multigene family of xenobiotic metablizing isoenzymes (e.g., GSTA1 having Genbank Accession No. LI 3269, GSTA2 having Genbank Accession No. NM000846, GSTA3 having Genbank Accession No. AF020919, GSTA4 having Genbank Accession No. NM001512, GSTM5 having Genbank Accession No. L02321, GSTα1 having Genbank Accession No. NM008181, GSTα2 having Genbank Accession No. NM008182, GSTα4 having Genbank Accession No. NM010357, etc.), and B-cell leukemia/lymphoma 2 genes, a class of genes that are mammalian homologues of ced-3, the “anti-death” gene of caenorhabtidis elegans (e.g., Bcl-2 having Genbank Accession No. NM000633, etc.).

Bcl-2 is a preferred polynucleotide. Bcl-2, the 26 kDa product of the Bcl-2 protoncogene, the mammalian homologue of ced-3, the ‘anti-death’ gene of caenorhabtidis elegans is a multifunctional protein (Reed, Nature, 387:773-776 (1997); and Chao et al., Ann Rev Immunol., 16:395-419 (1998)) that is localized to different intracellular membranes including those of the mitochondrion, endoplasmic reticulum, and the nucleus (Baffy et al. J. Biol. Chem., 268:6511-6519 (1993); Lam et al., Proc. Natl. Acad. Sci., USA, 91:6569-6573 (1994); and Ryan et al., Proc. Natl. Acad. Sci., USA, 91:5878-5882 (1994)), which coincidentally are also the major intracellular sites of production of the reactive oxygen intermediates (ROI). Bcl-2, generally considered a custodian of mitochondrial functional integrity, stabilize mitochondrial membrane by preventing the release of cytochrome c to the cytosol, thus blocking caspases activation. Translocation of cytochrome c shunts electrons from the electron transport chain and ATP generation to oxygen radical production (Cai et al., Biochim Biophys Acta., 1366:139-149 (1998); and Esposti et al., J. Biol. Chem., 274:29831-29837 (1999)) and initiates the apoptotic cascade.

Bcl-2 has been shown to facilitate repair and recovery from mitochondrial (mt) DNA damage and decrease apoptosis in different cell lines (Ryan et al., Proc. Natl. Acad. Sci., USA, 91:5878-5882 (1994); Hockenbery et al., Cell, 75:241-251 (1993); and Deng et al., Exp. Neurol., 159:309-318 (1999)). Cellular survival mediated by Bcl-2 protein family members implicate mechanisms that relate to their capacities to promote ion channel formation, protein binding, and increase the levels of reducing species, especially those of reduced glutathione (GSH) (Ellerby et al., J. Neurochem., 67:1259-1267 (1996)) and influence its intracellular distribution (Voehringer et al., Free Radic. Biol. Med., 27:945-950 (1999)).

Specific conditions for the uptake of a polynucleotide are well known in the art. They include, but are not limited to, retroviral infection, adenoviral infection, transformation with plasmids, transformation with liposomes containing exogenous nucleic acid, biolistic delivery (i.e., loading the polynucleotide onto gold or other metal particles and shooting or injecting into the cells), adenoassociated virus infection, and Epstein-Barr virus infection. These may all be considered “expression vectors” for the purposes of the invention.

The expression vectors may be either extrachromosomal vectors or vectors which integrate into a host genome. Generally, these expression vectors include transcriptional and translational regulatory factors operably linked to a coding region for the polypeptide. In general, the transcriptional and translational regulatory factors may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences. Promoter sequences encode either constitutive or inducible promoters. The promoters may be either naturally occurring promoters or hybrid promoters. Hybrid promoters, which combine elements of more than one promoter, are also known in the art, and are useful in the present invention. In addition, the expression vector may include additional elements. For example, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art. Suitable retroviral vectors include LNL6, LXSN, and LNCX. Suitable adenoviral vectors include modifications of human adenoviruses such as Ad2 or Ad5, wherein genetic elements necessary for the virus to replicate in vivo have been removed.

The conditions effective for uptake of the polynucleotide will depend on its form. Thus, for example, when it is in the form of an adenoviral, retroviral, or adenoassociated viral vector, the effective conditions are those that allow viral infection of the cell. In some embodiments, the exogenous nucleic acid is incorporated into the genome of the target cell. These conditions are generally well known in the art.

Additionally, the methods described herein can be used to create cell lines and animal models that can be used for drug screening and therapy.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. The references cited herein are expressly incorporated by reference.

### EXAMPLES

### Example 1

### Glutathione-S-transferase Protects Retinal Pigment Epithelial Cells from Oxidative Stress

Gene Transfer Methods.

Primary or SV-40 transformed human RPE cells grown to 85% confluency are mixed with GST plasmid DNA and TRANSFAST reagent (Promega, Madison, Wis.) in DMEM medium without serum and incubated for 1 hour (h) at 37° C. The cells are then overlayed with DMEM with 10% fetal calf serum and incubated for 48 hours. Following this G418 antibiotic is adding in concentrations ranging from 200-800 micrograms per milliliter (μg/ml). Over the next 14 days, surviving clones are selected and grown in flasks.

Experimental Design.

These studies have demonstrated increased baseline GST activity compared to a vector only transfected and wild type RPE cell lines (Table 1). GST expression in transfected cells was also confirmed by Western blotting. Studies were conducted to determine if enhanced GST activity would protect against mitochondrial functional decline using the MTT assay. Previous studies have shown a close correlation between mitochondrial functional decline and mtDNA damage. Plates of SV-40 transformed human RPE cells (n=5, each group) grown to 80-90% confluency which were transfected with mGST A1-1, or vector were exposed to hydrogen peroxide (25-200 μμM) or cumene hydroperoxide (62.5-1000 micromolar (μM)) for 1 hour at 37° C. in a CO₂ incubator. The cells were then harvested for the MTT.

TABLE 1
GST enzyme activity in mGST A1-1 and A2-2 transfected RPE cells

| | RPE cells | | | |
|---|---|---|---|---|
| | control | mGST A1-1 | mGST A2-2 | Vector |
| GST activity | 0.035 | 0.058 | 0.055 | 0.031 |
| (units/min/mg | | | | |
| protein) towards | | | | |
| CDNB | | | | |

Results.

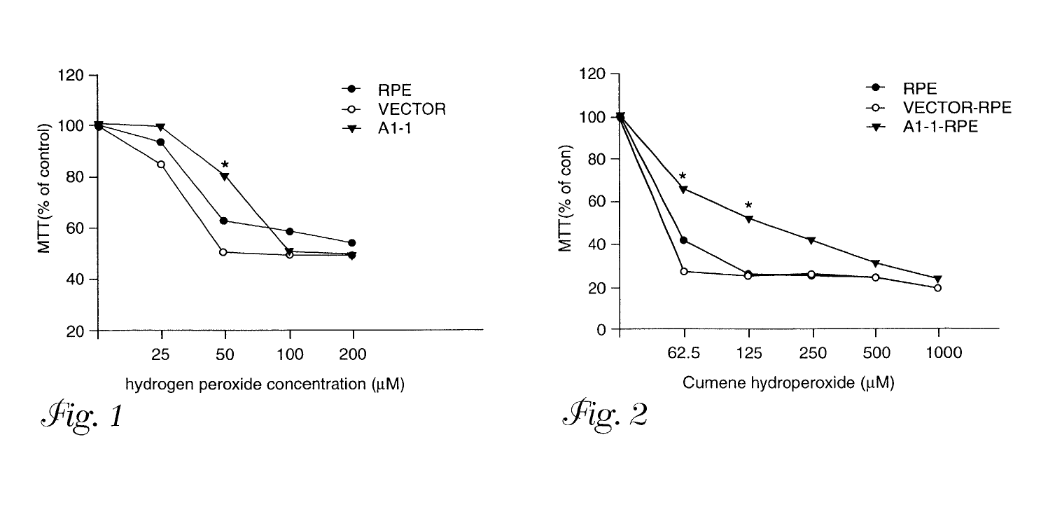

Hydrogen peroxide exposed (50 μM) mGST A1-1 transfected RPE cells retained more mitochondrial function (29%, p<0.05) compared to nontransfected control cells (FIG. 1). Similarly, cumene hydroperoxide treated mGST A1-1 transfected RPE cells had a significant protection (58% and 100%, p<0.05) at the 62.5 and 125 μM doses, respectively (FIG. 2). Additionally, mGST A1-1 transfected RPE cells demonstrated less cell death (56% and 43%) after exposure to 50 and 100 μM hydrogen peroxide (data not shown). These results support the hypothesis that GSTs are important in protecting RPE cells from oxidative stress.

### Example 2

### Bcl-2 Overexpression Increases Survival in Human Retinal Pigment Epithelial Cells Exposed to H₂O₂

Materials.

Electrophoretic-grade reagents: sodium chloride, sodium deoxycholate, sodium dodecyl sulfate (SDS), ethylene glycol-bis (β-aminoethyl ether)-N,N,N′,N′-tetraacetic acid (EGTA), NP-40, polyacrylamide, Tris, Tween-20, and non-fat dry milk were obtained from Biorad Laboratories (Hercules, Calif.). Nitrocellulose membranes were obtained from Micron Separations (Westborough, Mass.). Polyclonal antibody raised to Bcl-2, was purchased from Santa Cruz Biotechnology (Santa Cruz, Calif.). The electrochemiluminescence (ECL) Western Blotting kit including horseradish peroxidase-conjugated secondary antibody was obtained from Amersham-Pharmacia Biotechnology (Piscataway, N.J.). Goat anti-rabbit secondary antibody was used with the Bcl-2 antibody. 3-[4,5-dimethylthiazol-2-yl]-2,5-diphenyl tetrazolium bromide (MTT) was purchased from Promega (Madison, Wis.). Ethylenediaminetetraacetic acid (EDTA) dimethyl sulfoxide (DMSO) and sucrose were purchased from Sigma-Aldrich Co. (St. Louis, Mo.). Bradford reagent was purchased from Biorad Laboratories (Hercules, Calif.). Gene PORTER 2 transfection reagent was purchased from Gene Therapy Systems, Inc. (San Diego, Calif.). Human (h) Bcl-2 cDNA in pSFFV-neo expression vector was kindly donated by Dr. Stanley J. Korsmeyer, Howard Hughes Medical Institute, Washington University School of Medicine (St. Louis, Mo.). APOALERT kits were obtained from Clontech, Laboratories Inc., Palo Alto, Calif.).

RPE Cell Cultures and Bcl-2 transfection. SV40-transformed fetal male human RPE cells (RPE 28 SV4, Coriell Institute, Camden, N.J.) were cultured as described by Ballinger et al., Exp. Eye Res., 68:765-772 (1999). The rationale for using this established and well-documented cell line was as follows. A disadvantage with primary cell cultures derived from different donors is that the cellular response to H₂O₂ may lack consistency, which would not be a concern with a well-defined cell line, such as the one used in these studies. These transformed cells exhibit epithelioid morphology and retain physiological functions characteristic of primary human RPE cells, such as polarity and the ability to phagocytize rod outer segments. Additional studies also indicated that both primary and transformed cell lines respond similarly to oxidative stress (Kerendian et al., Curr. Eye Res., 11:385-396 (1992); and Kvanta et al., Ophthalmic Res., 26:361-367 (1994)).

Cells were plated the day before transfection, so that they would attain near confluency on the day that they were transfected with hBcl-2 cDNA in pSFFV-neo expression vector or the same vector in which the Bcl-2 cDNA had been deleted. Hydrated Gene PORTER 2 reagent was diluted with serum-free, phenol-red free Minimal Essential Media (MEM, GIBCO, Grand Island, N.Y.). The hBcl-2 cDNA in pSFFV-neo expression vector (Hockenbery et al., Nature, 348:334-336 (1990)) was appropriately diluted with the proprietary provided DNA diluent according to the manufacturer's instructions and was incubated for 2-10 minutes (min) at room temperature. Then the reagents were mixed and incubated for 5-10 min at ambient temperature. RPE cells were screened for Bcl-2 expression by western blot analysis as described below.

Western blot analysis. Non-transfected (control) cultured RPE cells and cells that had been transfected with both Bcl-2 and/or pSFFV-neo expression vector only were lysed by RIPA buffer (1 mM Tris-HCl, sodium deoxycholate, NaCl, EGTA, 1% NP-40 (Rao, Oncogene, 13:713-719 (1996)). Cell lysates corresponding to 50 micrograms (μg) protein, determined according to Bradford, Anal. Biochem., 72:248-254 (1976), and pre-stained molecular weight markers were loaded onto 1% sodium dodecyl sulfate (SDS), 10-15% polyacrylamide gels (PAGE) under reducing conditions. Following electrophoretic separation, the proteins were transferred to nitrocellulose membranes. After blocking with 5% non-fat dry milk and washing in 10 mM Tris-buffered saline, pH 7.5, containing 0.1% Tween-20 (T-TBS), the membranes were incubated with the appropriately diluted antibody to Bcl-2, for 3 h at room temperature. The primary antibody was decanted and the membranes were washed with TBS. The membranes were then incubated for 2 h at room temperature with horseradish peroxidase conjugated secondary antibodies (goat anti-rabbit for the polyclonal Bcl-2 antibody) in T-TBS diluted to the same concentrations as the primary antibody. The membranes were washed with TBS and incubated with the ECL substrate for 1 min and then exposed to X-OMAT AR film (Eastman Kodak, Rochester, N.Y.). The processed gel images were analyzed using a phosphoimager with the supplied ImageQuant software (Molecular Dynamics, Sunnyvale, Calif.).

Hydrogen peroxide treatments. Hydrogen peroxide (H₂O₂) was diluted with MEM and the concentration was determined according to Iyer et al., Nature, 192:535-541 (1961). Monolayer cultures (80-85% confluent) of non-transfected control, Bcl-2-tranfected or mock-transfected RPE cells (the pSFFV-neo expression vector with the Bcl-2 cDNA deleted) in serum-free, phenol red-free MEM were exposed to 50-200 μM H₂O₂ for 1 h as previously described in Ballinger et al., Exp. Eye Res., 68:765-772 (1999) and H₂O₂ concentrations were monitored according to Nowak, Biomed. Biochim. Acta., 49:353-362 (1990).

MTT Assay.

The MTT reduction assay which measures mitochondrial redox function was used as the index for cell survival. Near confluent cells that were non-transfected controls or cells that had been transfected with either Bcl-2, or just the vector (mock-transfected) were seeded onto 96-well plates at a density of 12000 cells per well. Forty-eight hours later, the medium was replaced with one that was serum-free containing different concentrations of H₂O₂ (50-200 μM). Cells were exposed to H₂O₂ for 1 h, the media containing the H₂O₂ was removed and the cells were washed with phosphate buffered saline (PBS). The cells were then incubated with serum-free medium containing 4.83 μM MTT for 2 h at 37° C. Four hours later, 150 μl DMSO was added to each well and the plates were shaken for 10 min. The supernatants were removed into fresh multi-well plates and the optical densities of the solutions were read at 590 nanometers (nm) (Hansen et al., J. Immunol. Methods, 119:203-210 (1989)) using a scanning multi-well spectrophotometer (Amersham-Pharmacia Biotech, Piscataway, N.J.). Absorbance values were converted to MTT reduction as previously described in Ballinger et al., Exp. Eye Res., 68:765-772 (1999). Values were normalized with respect to the untreated control cultures not exposed to H₂O₂ (100% mitochondrial redox function). Determinations were performed in duplicate and experiments were repeated (n=4).

DNA Damage.

Quantitative polymerase chain reaction (QPCR) was used to determine the average lesion frequency per DNA strand in specified sequences for both template strands (Ballinger et al., Exp. Eye Res., 68:765-772 (1999); Kalinowski et al., Nucleic Acids Res., 20:3485-3494 (1992); and Van Houten et al., Mutat. Res., 403:171-175 (1998)). Only intact DNA sequences were amplified and lesions including strand breaks, apurinic sites and base modifications caused by H₂O₂ damage would prevent amplification. To control for template copy number differences, and damage unrelated to experimental conditions, DNA sequences of approximately 200 kb base pairs were quantitatively amplified. Total RPE cellular DNA from control cells and cells transfected with either Bcl-2 or the vector only were isolated using a genomic tip 20G kit (Quiagen, Inc., Valencia, Calif.) according to the instructions. Total DNA concentrations were determined by ethidium bromide fluorescence using an A4-filter fluorimeter with an excitation band pass filter at 360 nm and emission cut-off filter at 600 mn (Optical Devices, Elmsford, N.Y.) using λ/Hind III DNA as standard. Sample quality was assessed by QPCR of a 222 base pair mitochondrial (mt) DNA fragment and an 84 base pair fragment of the nuclear (n) β-globin gene as previously described by Ballinger et al., Exp. Eye Res., 68:765-772 (1999). QPCR was performed in a GeneAmp PCR system 9600 with the GeneAmp XLPCR kit (Perkin-Elmer, San Jose, Calif.). Reaction mixtures contained 15 nanograms (ng) genomic DNA as a template. The composition and sequences of the primers and the PCR conditions employed for the 16.2 kb mtDNA, and the 13.4 kb β-globin products were as previously described by Ballinger et al., Exp. Eye Res., 68:765-772 (1999). A quantitative control using a 7.5 ng genomic template was included in every PCR series. PCR products were electrophoretically resolved on 1% agarose gels (dissolved in TBE buffer, Amresco, Solon, Ohio) at 80-90 v for 4 h. Dried gels were exposed to phosphor screens for 12 h and quantitated using ImageQuant software (Molecular Dynamics, Sunnyvale, Calif.). DNA lesion frequencies (λ) were calculated as the amplification of damaged DNA relative (A_{d}) to non-damaged controls (AO), where λ=−In A_{d}/A₀. Experiments to assess DNA damage in genomic and mtDNA were determined three times in duplicate.

Statistical Analysis.

Data for the QPCR and MTT reduction assays are presented as the means ±standard deviation (SD). The student's t-test was used for statistical evaluation between experimental groups. When the p values were <0.05, values were considered significant.

Results: Confirmation of Bcl-2 Transfection in RPE Cells.

A western blot (not shown) confirmed that Bcl-2 transfection was successfully achieved, as Bcl-2 overexpression was shown in Bcl-2-transfected RPE cells compared to both non-transfected control cells and mock-transfected cells that had been transfected with the pSFFV-neo expression vector alone. Bcl-2 expression was not seen in non-transfected control and mock-transfected cells as the brief film exposure time was inadequate to reveal basal protein levels. Densitometric analysis of Bcl-2 expression in transfected RPE cells was shown to be enhanced compared to control and vector-only transfected cells.

Results: Mitochondrial Redox Function.

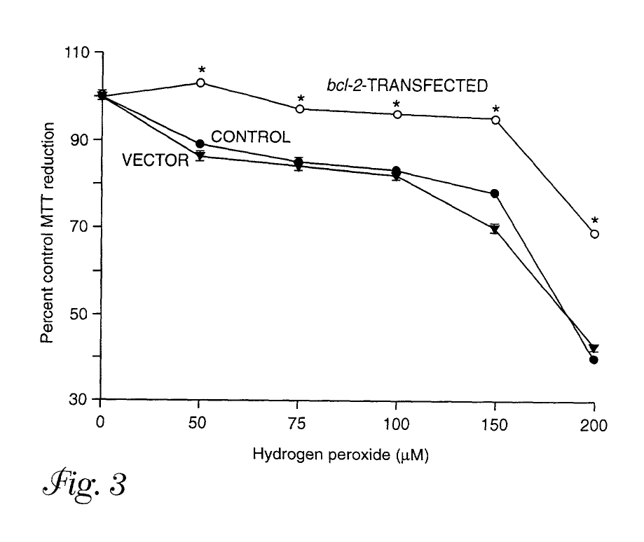

FIG. 3 shows that Bcl-2 overexpression protects RPE cells against H₂O₂-induced mitochondrial dysfunction, which causes cell death. Mitochondrial redox function in non-transfected cells (filled circles) was not significantly different from that in cells that had been transfected with the vector alone (filled triangles) and in both cell cultures, mitochondrial dysfunction and therefore cellular viability progressively declined with increasing concentrations of H₂O₂. Whereas, in the Bcl-2-transfected cells (open circles) redox function and cellular viability declined by only 25% with 200 μM H₂O₂, compared to a 60% decline in non-transfected and mock-transfected cells.

Results: Mitochondrial DNA Damage.

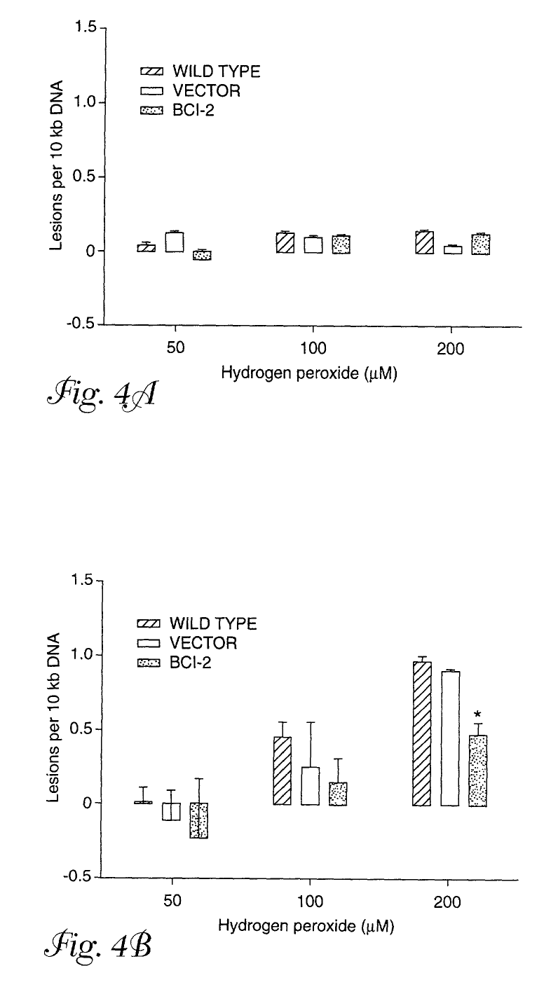

Damage to n and mt DNA, assessed by QPCR assays, in control, mock-transfected, and Bcl-2-transfected RPE cells exposed to different concentrations of H₂O₂, relative to cells not treated with H₂O₂ (zero class lesion frequency) is respectively shown in FIGS. 4A and 4B. MtDNA, represented by the 16.2 kb PCR product, was preferentially damaged by H₂O₂ treatment, whereas nDNA, represented by the 13.4 kb p-globin PCR product, was not damaged by even 200 μM H₂O₂, the highest concentration used. MtDNA was not significantly compromised in cells that were treated with 50 μM H₂O₂. With higher concentrations there was a trend for mtDNA to be increasingly damaged in control cells and cells that been vector only-transfected. RPE cells transfected with Bcl-2 were protected from H₂O₂-induced damage. Even with 200 μM H₂O₂ the lesion frequency per 10 kb DNA in Bcl-2-transfeted cells was 0.47±0.08 which was approximately 50% less than in comparable control (0.96±0.04) and vector only-transfected cells (0.90±0.01).

Discussion.

These studies show a dose-dependent mitochondrial dysfunction, which results in decreased cell survival, as determined by the MTT assay. Selective mtDNA damage was observed in response to H₂O₂ in control and mock-transfected cells, whereas the cells that overexpressed Bcl-2 retained more mitochondrial function and suffered less mtDNA damage, resulting in increased cellular viability. Deng et al., Exp. Neurol., 159:309-318 (1999) also recently showed increased survival of PC 12 cells overexpressing Bcl-2 than vector-only transfected cells after treatment with peroxynitrite, the product of H₂O₂ and nitric oxide using the MTT assay as the index for cell survival. They also reported that the transfected cells survived H₂O₂ exposure, that mtDNA damage was suppressed, and that the Bcl-2 overexpression facilitated mtDNA recovery after both H₂O₂ and peroxynitrite exposure. In this study it is also demonstrated that Bcl-2 transfection with consequential Bcl-2 overexpression rescues RPE cells from mtDNA damage, impaired mitochondrial respiratory function and apoptosis induced by H₂O₂. These studies suggest that Bcl-2 gene overexpression protects RPE cells from ROI exposure.

The complete disclosures of all patents, patent applications, publications, and nucleic acid and protein database entries, including for example GenBank accession numbers and EMBL accession numbers, that are cited herein are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

What is claimed is:

1. A method for generating a genetically engineered retinal pigment epithelial cell, the method comprising contacting a retinal pigment epithelial cell with a polynucleotide that encodes a glutathione-S-transferase under conditions effective for the uptake of the polynucleotide into the retinal pigment epithelial cell.

2. The method of claim 1 which is carried out in vivo.

3. The method of claim 1 wherein the polynucleotide is incorporated into an expression vector.

4. The method of claim 3 wherein the expression vector is selected from the group of a retrovirus, an adenovirus, a plasmid, and an adenoassociated virus.

5. A method for inhibiting the degeneration of a retinal pigment epithelial cell, the method comprising incorporating an exogenous polynucleotide into a retinal pigment epithelial cell, wherein the exogenous polynucleotide encodes a polypeptide useful in the inhibition of the degeneration of retinal pigment epithelial cells.

6. The method of claim 5 which is carried out in vivo.

7. The method of claim 5 wherein the polynucleotide is incorporated into an expression vector.

8. The method of claim 7 wherein the expression vector is selected from the group of a retrovirus, an adenovirus, a plasmid, and an adenoassociated virus.

9. The method of claim 5 wherein the polypeptide is a glutathione-S-transferase enzyme.

10. The method of claim 5 wherein the polynucleotide is selected from the group of a glutathione-S-transferase polynucleotide, a glutathione peroxidase polynucleotide, and a Bcl-2 polynucleotide.

11. A method for treating age-related macular degeneration in a subject, the method comprising incorporating an exogenous polynucleotide into a retinal pigment epithelial cell, wherein the exogenous polynucleotide encodes a polypeptide useful in the treatment of age-related macular degeneration.

12. The method of claim 11 wherein the polynucleotide is incorporated into an expression vector.

13. The method of claim 12 wherein the expression vector is selected from the group of a retrovirus, an adenovirus, a plasmid, and an adenoassociated virus.

14. The method of claim 11 wherein the polypeptide is a glutathione-S-transferase enzyme.

15. The method of claim 11 wherein the subject is a mammal.

16. The method of claim 15 wherein the subject is a human.

17. The method of claim 11 wherein the polynucleotide is selected from the group of a glutathione-S-transferase polynucleotide, a glutathione peroxidase polynucleotide, and a Bcl-2 polynucleotide.

18. The method of claim 11 which is carried out in vivo.

19. A retinal pigment epithelial cell comprising an exogenous polynucleotide that encodes a glutathione-S-transferase.

20. A method of delivering a polynucleotide to a subject's eye, the method comprising delivering the polynucleotide to the subretinal space of the subject's eye.

21. The method of claim 19 wherein the polynucleotide is delivered by injection of a liquid comprising the polynucleotide.

22. A method of inhibiting damage to a retinal pigment epithelial cell upon exposure to reactive oxygen intermediates, the method comprising incorporating a Bcl-2 gene into a retinal pigment epithelial cell.

23. A method of inhibiting damage to mitochondrial DNA in a retinal pigment epithelial cell upon exposure to reactive oxygen intermediates, the method comprising incorporating a Bcl-2 gene into a retinal pigment epithelial cell.
